(19) United States
(12) Patent Application Publication
(10) Pub. No.: US 2019/0116748 A1
(43) Pub. Date: Apr. 25, 2019

(54) APPLICATIONS OF ROOT-ADMINISTERED CHEMICAL HYBRIDIZING AGENTS IN PLANT BREEDING

(71) Applicant: BAYER CROPSCIENCE AKTIENGESELLSCHAFT, Monnheim am Rhein (DE)

(72) Inventors: Elmar Alfons Weissmann, Gatersleben (DE); Hans Guenter Welz, Leverkusen (DE)

(21) Appl. No.: 16/098,602

(22) PCT Filed: Apr. 26, 2017
(86) PCT No.: PCT/EP2017/059889

§ 371 Date: Nov. 2, 2018

(30) Foreign Application Priority Data
May 3, 2016 (EP) 16168109.3

(51) Int. Cl. A01H 1/06

(52) U.S. Cl. CPC A01H 1/06

(57) ABSTRACT

A method of applying a Chemical Hybridization Agent (CHA) to a plant. The method includes providing a CHA source and bringing the CHA source in contact with a root of the plant. The method further includes having the root take up a part of the CHA. The disclosure further relates to methods of producing a haploid and/or doubled-haploid plant or hybrid seed. The disclosed method may be used in various aspects of plant breeding.

Plant breeding typically involves the crossing of two genotypes. Crossing means that a plant from one specific genotype is emasculated to prevent unwanted cross or self-pollination and subsequently the remaining female organs are fertilized with pollen from another, genetically different plant. The crossings often follow different schemes depending on the aim of the crossing program or the research purpose. Crossing is instrumental in plant breeding programs such as: introducing or enhancing genetic variability from germplasm sources, producing (experimental) hybrid varieties, producing haploid or doubled-haploid lines, reverse breeding, recurrent backcrossing, marker-assisted selection etc.

Especially for cereals, most of which are monoecious and self-pollinating, the production of haploid or doubled-haploid plants has proven to be difficult, cumbersome and costly, whereas efficiency has been low. A limiting step has always been the emasculation of the plants to prevent cross or self-pollination.

While pollen is abundant and in most crops easily transferred (even by wind), manual emasculation is very labor-intensive due to flower morphology. In wheat, for example, each ear has to be reduced to approximately 20 spikelets. One spikelet is surrounded by glumes and can consist of more than 5 florets. The spikelets are reduced by hand excision to the two main florets. The lemma and palea of these florets are clipped to facilitate reaching into the florets. Three male organs (immature anthers) per floret have to be excised with tweezers. Care has to be taken to find all three anthers and to excise the anthers completely. Care must also be taken not to damage the female organs (ovule and stigma). After emasculation the ear is bagged. This procedure takes approx. 10 minutes per plant. A trained person can manually emasculate approx. 10 ears per hour.

In the past, several approaches were suggested and practiced for the induction of male sterility in plant species. Exemplary methods include manual emasculation, systems for the induction of male sterility based on cytoplasmic-genetic male sterility, the introduction of transgene(s) for the synthesis of cytotoxic or cytostatic polypeptides, the introduction of a transgene for the formation of an exogenous double-stranded RNA for the induction of RNA interference and the chemical induction of male sterility. Chemical sterilization has, inter alia, the advantage that no complex genetic engineering and long-term input of a male-sterile cytoplasm with backcrossing is needed. Drawbacks include that fact that most chemical hybridizing agents (CHA) are toxic and extreme care with handling is needed. Furthermore, spraying the chemicals always carries the risk of contaminating the user or the environment.

Therefore, there is the need for a safe and efficient method for producing male sterile plants to enhance the efficacy of any targeted pollinations (crossings), e.g. for developing line varieties, haploidy-mediated inbred lines or parental lines for hybrid varieties.

Accordingly, in a first aspect, the present invention relates to a method of applying a CHA to a plant comprising the steps of (a) Providing a source comprising at least one CHA; (b) Bringing said source in contact with at least one root of a plant and (c) Having the at least one root take up at least a part of said at least one CHA.

Chemical hybridization agents (CHA) are synthetic substances which, applied at a precise development stage of a plant, disturb the formation of pollen grains and thus render the plant “male sterile”. The sterilized plant may therefore only be fertilized by the pollen of another plant. Several CHAs have been developed in the course of time. Whereas in principle any CHA may be used in the methods of the present invention, preferred CHAs to be used in connection with the present invention are listed further below.

The term “source” as used in connection with the present invention denotes a medium in which the CHA may be provided such that it is also accessible to at least one root of a plant. The source may be a water reservoir where the plant roots are placed in, such as a hydroponic cultivation system, as well as a container comprising an aqueous solution in combination with an appropriate substrate. A source may also be an aqueous solution comprising the CHA which is applied to the at least one root at the appropriate time, e.g. by watering a plant comprised in a standard planting pot, edge length 12 cm, height 13 cm with 200 ml tap water solution.

Contact has to be made with at least one root of a plant, preferably with more than one root and most preferably with the complete root system of said plant in order to provide for efficient uptake of the CHA within as short a time as possible.

Bringing in contact may be effected in various ways including adding the CHA in the desired concentration to the source already in contact with the at least one plant root or placing the at least one plant root (together with the plant) into a source already comprising the CHA in the appropriate concentration.

In the course of the present invention it was surprisingly found that CHAs can be applied to and taken up by the roots of plants. Thereby, the need to spray plants or plant parts can be avoided. This alternative application technique for CHAs enables the easy, safe and convenient use of CHAs in all different applications where the induction of male sterility is a prerequisite for any cross pollination scheme (either pollination with pollen of a different variety or with genetically distinct species), as it may be required in the production of haploid and doubled-haploid plants and also the production of hybrid seed and/or plants. In particular, the efficiency of emasculation of plants having flowers carrying both male and female organs in plant breeding can be enhanced. Also, the efficiency of crossing and the creation of genetic variability are greatly increased by this novel technique.

In a second aspect, the present invention relates to a method of inducing male sterility in a plant, comprising carrying out the steps comprised in the method of applying a CHA to a plant as described above, wherein said step (b) of bringing in contact is effected prior to flowering of said plant.

Male sterility is the failure of plants to produce functional anthers, pollen and/or male gametes resulting in the incapability of plants to produce or release functional pollen grains. Besides manually achieved male sterility (effected by emasculation), five types or plant-inherent male sterility can be distinguished: 1) Genic male sterility, 2) Cytoplasmic male sterility, 3) Cytoplasmic-genic male sterility, 4) Chemically-induced male sterility and 5) Transgenic male sterility.

Several development scales for crop plants exist. For cereals, the most widely used one is the decimal Zadoks scale (J. C. Zadoks, T. T. Chang, C. F. Konzak, “A Decimal Code for the Growth Stages of Cereals”, Weed Research 1974 14:415-421) dividing cereal development into 92 stages. In order to efficiently induce male sterility, the CHA is applied at the latest at Zadoks stage 65 (for monocotyledonous plants) and may be applied already at Zadoks stage 01 and thereafter until stage 59 (the latter for dicotyledonous plants) which is immediately prior to flowering. The optimal timing of CHA application depends to a certain extent on the type of CHA used. For example, the preferred application time in the case of clofencet (2-(4-chlorophenyl)-3-ethyl-5-oxo-2,5-dihydropyridazine-4-carboxylic acid) ranges from stages 29 to 31, whereas azetidine-3-carboxylic acid is best applied between stages 57 and 60.

The development scale for crops other than cereals is the BBCH-scale, which is adapted according to crop/plant and in its structure based on the Zadoks scale. It can be reviewed in the monograph “Growth stages of mono- and dicotyledonous plants” (edited by Uwe Meier, Federal Biological Research Centre for Agriculture and Forestry, Germany), retrievable under http://www.jki.bund.de/fileadmin/dam_uploads/_veroeff/bbch/BBCH-Skala_englisch.pdf (last checked 21 Apr. 2016)

The principle underlying the application of a CHA in order to induce male sterility is that the CHA needs to be present in the target organs upon pollen development. In general, for monocotyledonous plants, application of a CHA is effected between BBCH 31 and BBCH 65, preferably between BBCH 31 and BBCH 60, whereas for dicotyledonous plants, a CHA is usually applied between BBCH 51 and 59.

Uptake of the CHA by the plant root system may depend on the substrate used for cultivation and on root penetration of the substrate used. Suitable substrates are e.g. soil, sand, non-natural culture substrates like vermiculite or simply water. For the case of solid substrates the amount of liquid containing the CHA applied to the substrate is chosen such that the liquid is completely adsorbed by the substrate thereby creating a depot for said CHA. After application of the CHA, irrigation plans preclude leaching of the CHA.

Application ranges of CHAs are estimated to be between about 1 mg/plant and about 250 mg/plant. Exemplary ranges include those between about 1 mg and about 220 mg/plant, such as between about 1.4 and about 216 mg/plant, about 2 mg/plant and about 200 mg/plant, such as about 5, about 10, about 15, about 20, about 25, about 50, about 75, about 100, about 150, about 175 mg per plant and any value in between those values.

The method of the invention is greatly facilitating the production of novel plant varieties because the natural variation occurring in the first generation (F1) after crossing may be immediately rendered into homozygous plants (e.g. by haploid or doubled-haploid techniques) having a specific genotype. In this way, repeated and time-consuming crossings can be avoided. This aspect of the invention may also be used in the production of hybrid seed, which results in a plant which is suitable for use as a parent in hybrid plant production. Multiple crossings are also part of breeding schemes which involve Mapping Quantitative Trait Loci (MQTL), Bulked Segregant Analysis (BSA) or Reverse Breeding.

In another aspect, the invention relates to a method of producing haploid embryos or plants comprising carrying out the steps according to the method of inducing male sterility in a plant as described above, followed by (d) Fertilizing said plant with an appropriate pollen of a genetically remote plant (cf. D. A. Laurie and M. D. Bennett 1988: “The production of haploid wheat plants from wheat×maize crosses”, Theoretical and Applied Genetics 76: 393-397), thereby generating haploid embryos and (e) Optionally regenerating haploid plants from the haploid embryos generated in step (d).

Haploid embryos or plants are embryos or plants with only one set of chromosomes reflecting the gametic chromosome number. For diploid species such as barley, a haploid plant has a single chromosome set instead of two chromosome sets. For polyploid plants, the number of chromosome sets in the respective haploid plant is half of that of the natural diploid plant. For example hexaploid bread wheat may serve as the basis for producing haploid wheat plants comprising three chromosome sets, for durum wheat which is tetraploid, haploid plants comprise two chromosome sets.

Fertilizing with pollen of a genetically remote plant in connection with the present invention means having pollen of a genetically remote plant, e.g. maize being genetically remote from wheat, “pseudo-fertilize” the plant serving as the origin for haploid embryos. In this regard, pollen of a remote plant means pollen of a plant which under natural circumstance could not fertilize the flowers of the mother plant of the haploid embryos to be produced. In other words, the plant needs to at least be of a different species so that the chromosomes of the pollen would be unable to pair with the chromosomes of the mother plant of the haploid embryos. For cereals, pollen from a remote plant may originate from maize. Upon pollination of an emasculated wheat plant with maize pollen, the maize chromosomes are quickly eliminated and the development of haploid embryos is induced, with representation of only the female gametes.

Regenerating haploid embryos is a procedure well-known in the art and involves hormone treatment shortly, such as one or two days, after pollination, e.g. with the pollen of a remote plant. Suitable hormones include Dicamba (2,4-dichlorophenoxyacetic acid) and BAP (Benzyl-Amino-Purine). Afterwards the caryopses are excised, rescuing the endosperm-less embryos, and are allowed to germinate on a nutrient-containing agar medium to produce haploid plantlets (see e.g. Laurie and Bennett, 1988; Theor Appl Genet 76: 393-397). Alternatively, callus induction may be used to obtain plants from small, undifferentiated globular embryos to obtain regenerated plants.

In yet another aspect, the present invention relates to a method of producing doubled-haploid plants comprising applying the steps according to the method of producing haploid embryos or plants as described above, followed by (f) Treatment of the plants with an agent causing at least the doubling of the chromosome set of said embryo or plant, such as colchicine or N₂O (nitrous oxide).

A doubled haploid (DH) is a genotype formed when haploid cells undergo chromosome doubling.

Any agent causing polyploidization, preferably chromosome doubling, may be used in connection with the present invention. The most prominent example of such agents is colchicine which induces chromosome doubling. However, also nitrous oxide may be used in this respect (see e.g. Dang et al., Inducer line generated double haploid seeds for combined waxy and opaque 2 grain quality in subtropical maize (Zea mays. L.); Euphytica 2012, 183:153-160).

A review of existing methods for producing doubled haploids is given in “Doubled Haploid Production in Crop Plants—A Manual” (Maluszynski, Kasha, Forster and Szareijko (Editors), Springer Science & Business Media, New York 2003, ISBN 978-90-481-6393-9) which is herewith incorporated by reference in its entirety.

In another aspect, the present invention provides for a method of producing a plant comprising (g) Selecting from a pool of doubled-haploid plants produced by the method of producing doubled-haploid plants as described above a plant suitable as plant variety. This aspect of the invention may also be used in the production of hybrid seed. Here, step (g) results in a plant which is suitable for use as a parent in hybrid seed production.

Accordingly, the present invention also relates to a doubled-haploid plant or a hybrid plant or hybrid seed generated by the method according to claim 4.

The present invention also relates to the use of the plant obtained by the method according to the invention in breeding, such as for mapping quantitative trait loci (QTL), backcross breeding, bulked segregant analysis, genetic maps, genetic studies, genomics, reverse breeding, crossing of plants (e.g. with the objective to create genetic variation) and variety development. In other words, the present invention also relates to a method for assisting in breeding comprising producing the haploid or doubled-haploid plant of the invention and subjecting said plant to further analysis using QTL mapping, backcross breeding, bulked segregant analysis, genetic maps, genetic studies, genomics, reverse breeding, crossing of plants (e.g. with the objective to create genetic variation) and variety development.

Mapping Quantitative Trait Loci (MQTL): Most of the economic traits are controlled by genes with small but cumulative effects. Although the potential of DH populations in quantitative genetics has been understood for some time, it was the advent of molecular marker maps that provided the impetus for their use in identifying loci controlling quantitative traits. As the quantitative trait loci (QTL) effects are small and highly influenced by environmental factors, accurate phenotyping with replicated trials is needed. This is possible with doubled-haploid organisms because of their true breeding nature and because they can be produced in large numbers.

Backcross breeding: In backcross conversion, genes are introgressed from a donor cultivar or related species into a recipient elite line through repeated backcrossing. The development of molecular markers combined with doubled haploidy provides a shorter cycle of selection based on the genotype (marker) rather than the phenotype. Already in the first backcross (BC) generation BC1, a genotype with the character of interest can be selected and converted into a homozygous doubled-haploid genotype.

Bulked segregant analysis (BSA): In bulked segregant analysis, a population is screened for a trait of interest and the genotypes at the two extreme ends form two bulks. Then the two bulks are tested for the presence or absence of molecular markers. Since the bulks are supposed to contrast in the alleles that contribute positive and negative effects, any marker polymorphism between the two bulks indicates the linkage between the marker and trait of interest. BSA is dependent on accurate phenotyping and the DH population has particular advantage in that they are true breeding and can be tested repeatedly.

Genetic maps: DH populations have become standard resources in genetic mapping for species in which DHs are readily available. Doubled-haploid populations are ideal for genetic mapping. It is possible to produce a genetic map within two years of the initial cross regardless of the species. Map construction is relatively easy using a DH population derived from a hybrid of two homozygous parents as the expected segregation ratio is simple, i.e. 1:1.

Genetic studies: Genetic ratios and mutation rates can be read directly from haploid populations. Doubled-haploid (DH) populations can be used, for example, to analyze the segregation of markers.

Reverse breeding: “Reverse breeding” (RB) is a novel plant breeding technique designed to directly produce parental lines for any heterozygous plant, one of the most sought-after goals in plant breeding. RB generates perfectly complementing homozygous parental lines through engineered meiosis. The method is based on reducing genetic recombination in the selected heterozygote by eliminating meiotic crossing over. Male or female spores obtained from such plants contain combinations of non-recombinant parental chromosomes which can be cultured in vitro to generate homozygous doubled-haploid plants (DHs). From these DHs, complementary parents can be selected and used to reconstitute the heterozygote “in perpetuity” (see Dirks et al., Plant Biotechnology Journal 2009, 7: 837-45).

Crossing of plants: Traditional breeding methods are slow and classical cultivar development typically takes 10-15 years. Another disadvantage is inefficiency of selection in early generations because of heterozygosity. These two disadvantages can be overcome by DHs, and more elite crosses can be evaluated and selected within less time.

Cultivar development: Uniformity is a general requirement of commercial line varieties in most species, which can be obtained through DH production. There are various ways in which DHs can be used in cultivar production. The DH lines themselves can be released as cultivars, they may be used as parents in hybrid cultivar production or more indirectly in the creation of breeders lines and in germplasm conservation.

Any preferred embodiment as described herein may be applied to each of the aspects of the present invention unless stated otherwise. Furthermore, preferred embodiments as described herein may be combined amongst each other as to be read in the claims appended hereto.

In a preferred embodiment, the plant is selected from cereals, fruits, vegetables, other crop plants and ornamentals.

Basically any plant, preferably any hermaphrodite plant, may be used in the present invention.

In a more preferred embodiment, said plant is a cereal plant.

A cereal is any grass cultivated for the edible components of its grain, composed of the endosperm, germ, and bran. Besides wheat, rye, rice, barley, oats, millet and triticale the cereals family also comprises maize, rice and fonio.

In an even more preferred embodiment said plant is selected from the group consisting of wheat (Triticum spp.), rye, rice, barley, oats, millet and triticale, more preferably wheat (Triticum spp.), rye, oats, millet and triticale.

Most preferably, said plant is wheat, such as Triticum aestivum L. and/or durum wheat T. durum.

In another preferred embodiment, essentially no other plant part is in contact with said CHA.

“Essentially no other plant part” in connection with the present invention denotes that the ratio of root surface in contact with a source comprising a CHA to surface of any other plant part such as stem, leaves, flowers is more than 9:1, preferably more than 19:1, more preferably 99:1.

In a preferred embodiment, the amount/concentration of CHA applied ranges between 1.4 mg/plant and 216 mg/plant.

In another preferred embodiment, said source is irrigation water.

In yet another preferred embodiment, said plant is cultivated in a substrate or as hydroponic culture.

In a preferred embodiment, said CHA is selected from 2-(4-chlorophenyl)-3-ethyl-2,5-dihydro-5-oxopyridazine-4-carboxylic acid (clofencet), 1-(4-chlorophenyl)-4-oxo-5-(methoxyethoxy) cinnoline-3-carboxylic acid (sintofen), azetidine-3-carboxylic acid (WL84811), 2-chloroethylphosphonic acid (Ethrel, Ethephon), 1-(4-chlorophenyl)-1,4-dihydro-6-methyl-4-oxopyridazine-3-carboxylic acid (fenridazon, also fenridazon potassium known as RH-0007 or Hybrex), DABCO (1,4-diazabicyclo[2.2.2]octane) and its quaternary salt derivatives (in particular halogen derivatives, such as DABCO-benzyl chloride and DABCO-BCL3), 2,4-Dichlorophenoxybutyric acid (Embutox), sodium 2,3-dichloroisobutyrate, triiodobenzoic acid, naphthalene acetic acid, maleic hydrazide, bromoxonil, glyphosate, giberrelic acid, iodosulfuron, flufenacet, Sogital (SC2053, Wong et al., 1995, Plant Growth Regulation 16, 243-48), DPX 3778 (see e.g. Theurer, Canadian Journal of Plant Science 1979; 59: 463-68) and nitroarylalkylsulfone derivatives or water-soluble salts thereof. A review of the use of various compounds as CHA is reviewed in “Chemical Hybridizing Agents”, Muhammad Boota Sarwar, retrievable under http://de.scribd.com/doc/62470606/Chemical-Hybridizing-Agents-Report1#scribd) and especially for cereals in “Hybrid breeding in wheat: technologies to improve hybrid wheat seed production” (R. Whitford et al., Journal of Experimental Botany 2013, doi:10.1093/jxb/ert333) which is herewith incorporated by reference in its entirety.

In a more preferred embodiment, said CHA is selected from clofencet, sintofen, azetidine-3-carboxylic acid, fenridazon, DABCO, bromoxonil, iodosulfuron and derivatives and salts thereof.

In a more preferred embodiment, said CHA is clofencet, sintofen or azetidine-3-carboxylic acid or a salt thereof. In an even more preferred embodiment, said CHA is clofencet, the potassium salt of clofencet or azetidine-3-carboxylic acid or a salt thereof.

In some embodiments, said CHA is clofencet, the potassium salt of clofencet or sintofen.

For root application in a field situation, the dose rate of CHA needs to be 2-3 times higher than for a foliar spray application, as was determined in preliminary experiments (see also discussion in the Examples section). Furthermore, because potted plants, with one plant per pot, in the experiments conducted carried four spikes (supernumerary spikes were removed), while typical field plants carry only 1.15 spikes (in a canopy with 400 spikes per square meter and 350 plants per square meter), the final dose rate of CHA when applied to potted plants via the roots was calculated to be approximately 10 times higher (see appended examples) but may as well be lower to achieve satisfying results.

Satisfying results are achieved if at least 85% sterility is present, preferably at least 90%, more preferably at least 95% and most preferably at least 98%.

For example, in order to achieve an efficient induction of male sterility by foliar application under field conditions, the agent clofencet needs to be applied between the growth stages Zadoks 32 and 39 (between tip emergence of the penultimate leaf and emergence of visible ligule of a flag leaf) in a dosage ranging from 0.79 to 1.55 mg per plant (value calculated based on a density of 350 plants per square meter). As described above, CHA concentrations need to be increased for various reasons for setups with potted plants. In the case of clofencet, the amount of CHA applied per plant thus ranges from about 5 mg to about 25 mg, preferably from about 7.5 mg to about 16 mg or any value in between these ranges.

Sintofen is applied in the field by a foliar spray when the length of a developing spike in the main stem is 14 mm to 18 mm, which corresponds to Zadoks stages 31 to 32, with a dose of at least 0.34 to 0.43 mg per plant (value calculated based on a density of 350 plants per square meter). Accordingly, it is expected that the dose to be applied in root application ranges between about 1.5 mg and about 6 mg, preferably about 1.5 to about 5 mg per plant, more preferably about 2 to about 4 mg per plant, or any number in between these ranges.

In another more preferred embodiment relating to clofencet or azetidine-3-carboxylic acid, the bringing into contact of said CHA in cereals is effected between Zadoks stages 31 and 65. The amount of azetidine-3-carboxylic acid applied per plant for root applications ranges from about 0.5 mg to about 5 mg per plant or any value in between these ranges.

The following examples illustrate the invention in a non-limiting fashion.

### EXAMPLE SETUP

Chemical Hybridization Agents (CHA) are synthetic substances which, applied at a precise development stage of a plant, disturb the forming of grains of pollen and thus render the plant “male-sterile”. The sterilized plant may therefore only be fertilized by the pollen of another plant. Several CHAs have been developed in the course of time. Examples 1 and 2 demonstrate the application of two different CHAs in different doses and ways of treatment of the roots of single plants of two varieties and the effect regarding male sterility. Example 3 demonstrates that the proposed CHA treatment used in Example 2 and the resulting sterility of the plants is an effect of the caused male sterility and has little or no negative effect on female fertility in terms of seed set.

Methods

The design of Example 1 and Example 2 is summarized in Table1

### Example 1

Wheat plants of the wheat varieties JB Asano and Apache were grown in the greenhouse in standard planting pots, bottom perforated, edge length 12 cm, height 13 cm, with one plant per pot. Twelve pots were placed into a plastic tray, bottom non-perforated with fleece inlay (e.g. available at Hermann Meyer KG unter article no. 44 54 11). Standard greenhouse growing substrate soil (bedding substrate 1 of Klasmann-Deilmann GmbH) was filled into the pot ca. 2 cm below edge. Plants were grown under standard growing conditions with respect to fertilizer, watering, disease and light management. Each plant was reduced to 4 tillers. The necessary active amount of Clofencet per plant was calculated according to the standard foliar spray application protocol of 5.42 kg per hectare assuming 400 ears per square meter taking the higher tiller number of potted plants into account (average number of ears per plant in the field is typically 1.15 but it was 4 in our potted plants, so the dose rate for root application per plant, i.e. per pot, was adjusted upward). At Zadoks growth stage 31 the plants were watered with 0.0 mg (control), 10.84 mg, 16.26 mg and 21.68 mg, respectively, of Clofencet in 200 ml tap water per pot. Each treatment was repeated on 12 plants, whereas the highest concentration was repeated on 24 plants. Before flowering time (Zadoks stage 57 of control group) ears were bagged to avoid unwanted cross-pollination. Bags were removed at beginning of grain filling (Zadoks stage 75 and higher), seed set was determined at kernel ripeness of the control group. When ears carried mature seeds, the ears were threshed with a hand-threshing device (Baumann Dreschhexe #4.701.200) and the kernels per plant were counted. A wheat ear is called completely sterile (=100% sterility) when no kernel developed on a bagged ear. The average number of grains per plant of the control plants was set to 0% sterility.

TABLE 1
Experimental design for Examples 1 and 2.

| | Example 1 | Example 2 |
|---|---|---|
| Factors | Clofencet | WL84811 |
| | 2-(4-chlorophenyl)-3-ethyl-2,5-dihydro-5- | azetidine-3-carboxylic acid |
| | oxopyridazine-4-carboxylic acid | |
| concentration 1 | 200 ml water (control) | 100 ml water (control) |
| concentration 2 | 200 ml water + 10.84 mg Clofencet | 100 ml water + 1 mg WL84811 |
| concentration 3 | 200 ml water + 16.26 mg Clofencet | |
| concentration 4 | 200 ml water + 21.68 mg Clofencet | |
| Varieties | Apache, JB Asano | KWS Target, JB Asano |
| Number of | 12 | 2 |
| replications | | |
| Number of treatments | 1 | 11 (every week one application) |
| per plant | | |
| Treatment stage | Zadok stage 31 | Zadok stages 31 to 65 |
| Growing Media | Bedding substrate1 | Sandy loam |
| | Klasmann-Deilmann GmbH | |

The results of Example 1 show that a single root application dose of 10.84 mg Clofencet (concentration 2) per plant with four tillers at growth stage 31 already gives a satisfying amount of average male sterility, and 16.26 mg (concentration 3) per plant are enough to ensure male sterility of almost 100%. Almost no plants watered once with an active dose of 16.26 mg of Clofencet or more in Zadoks stage 31 did set any seeds. At the lower concentration 2 a certain effect of the genotype (Apache vs. JB Asano) on average male sterility can be detected, which however is irrelevant at higher concentrations. Data not shown indicate that doses of active substance higher than 21.68 mg per plant do not have any further positive effect.

Results

TABLE 2
Results of Example 1

| | Clofencet | Water per | Number of | Number of | | Average sterility | |
|---|---|---|---|---|---|---|---|
| | per plant per | plant per | treatments per | plants treated | Treatment | relative to | |
| Example 1 | treatment | treatment | plant | (replications) | stage | control | |
| Variety | | | | | | Apache | JB |
| | | | | | | | Asano |
| Concentration | mg/plant | ml | | | Zadok stage | [%] | [%] |
| 1 (control) | 0 | 200 | 1 | 12 | 31 | 0 | 0 |
| 2 | 10.84 | 200 | 1 | 12 | 31 | 91.88 | 94.72 |
| 3 | 16.26 | 200 | 1 | 12 | 31 | 98.69 | 99.96 |
| 4 | 21.68 | 200 | 1 | 24 | 31 | 99.64 | 98.65 |

### Example 2

Wheat plants of the wheat varieties JB Asano and KWS Target were grown in the greenhouse in standard planting pots, bottom perforated, edge length 12 cm, height 13 cm, with one plant per pot. Twelve pots were placed into a plastic tray, bottom non-perforated with fleece inlay. Sandy loam from the field was filled into the pots ca. 2 cm below edge. Plants were grown under standard growing conditions with respect to fertilizer, watering, disease and light management. Each plant per pot was reduced to 4 tillers. The treatment was watering each pot with 0.0 mg (control) or 1.0 mg WL84811 in 100 ml tap water per pot. Each treatment was repeated on 2 plants per variety, watering with the treatment was done once a week for 11 weeks starting at Zadoks growth stage 31 until growth stage 65 of the control group. Ears were bagged to avoid unwanted cross-pollination. Bags were removed at beginning of grain filling (Zadoks stage 75 and higher), seed set was determined at kernel maturity of the control group. When ears had set seed, the ears were threshed by hand and the kernels per plant were counted. A wheat ear is called completely sterile (=100% sterility) when no kernel was developed on a bagged ear. The average number of grains per plant of the control plants was set to 0% sterility.

Results

TABLE 3
Results of Example 2

| | | | | Total | | | | |
|---|---|---|---|---|---|---|---|---|
| | WL84811 per | Water per | Number of | amount | Number of | | Average sterility | |
| | plant per | plant per | treatments | of | plants treated | Treatment | relative to control | |
| Example 2 | treatment | treatment | per plant | WL84811 | (replications) | stage | after CHA | |
| Variety | | | | | | | KWS | JB |
| | | | | | | | Target | Asano |
| Concentration | mg/plant | ml | | mg/plant | | Zadok stage | [%] | [%] |
| 1 (control) | 0.0 | 100 | 11 | 0.0 | 2 | 31 to 65 | 0 | 0 |
| 2 | 1.0 | 100 | 11 | 11.0 | 2 | 31 to 65 | 100 | 100 |

The result of Example 2 shows, that also a root treatment by regular weekly watering with small doses of 1.0 mg of WL84811 is very effective to generate male sterility. No plant treated with WL84811 and bagged before flowering set any seeds.

### Example 3

Male sterile plants generated by Clofencet treatment as described in Example 1 were used. Before single bagging the 4 ears of each plant at Zadoks stages 55-59, flowers were clipped to facilitate pollination. At flowering time (spikelets wide open) three male-sterile ears (no pollen in the transparent bags) were pollinated with pollen from an untreated control plant of the same wheat variety. One sterile ear was not pollinated to cross check the efficacy of the Clofencet treatment. Bags were removed at beginning of grain filling (Zadoks stage 75 and higher), seed set was determined at kernel maturity. If ears had set seed, the ears were threshed with a hand-threshing device (Baumann Dreschhexe #4.701.200) and the kernels per plant were counted. The average number of grains per plant of the control plants was set to 100% seed set.

Example 3 shows that the root treatment with Clofencet does not harm the female fertility of the treated plants. Plants treated once with Clofencet and pollinated with pollen of untreated control plants produced seed. Compared to the control with 100% seed set, Clofencet treatment of plants of the variety Apache even seems to enhance seed set.

Results

TABLE 4
Results of Example 3

| | | | | | | Average seed set of | |
|---|---|---|---|---|---|---|---|
| | | | | | | Clofencet treated | |
| | | | | | | plants after | |
| | Clofencet | Water per | Number of | Number of | | pollination with | |
| | per plant per | plant per | treatments | plants treated | Treatment | wheat pollen | |
| Example 3 | treatment | treatment | per plant | (replications) | stage | (rel. to control) | |
| Variety | | | | | | Apache | JB Asano |
| Concentration | mg/plant | ml | | | Zadok stage | [%] | [%] |
| 1 (control) | 0 | 200 | 1 | 12 | 31 | 100 | 100 |
| 2 | 10.84 | 200 | 1 | 12 | 31 | 101 | 98 |
| 3 | 16.26 | 200 | 1 | 12 | 31 | 115 | 94 |
| 4 | 21.68 | 200 | 1 | 24 | 31 | 107 | 75 |

### DISCUSSION

Examples 1 and 2 show that CHA applied to plants via root by watering is effective. This method is especially valuable in greenhouse applications, where waste water and soil recycling can be controlled. It prevents the greenhouse staff from aerosol pollution and limits the risk of unwanted exposure.

Example 1 shows, that even a single root application of CHA can be effective compared to the previously known foliar application. However, this single dose has to be a higher dose per plant compared to foliar application. One reason for this could be that the xylem-mediated transfer of the CHA from root to inflorescence is less effective than the leaf-to-spike phloem transport. The idea to soak the plants with a relatively high amount of solution (200 ml of water for a pot of 1728 ccm) and to retain the water with the diluted CHA within the growing media (soil or substrate), has the effect that the CHA may be successively incorporated into the plant over a period of time, thus making sure to hit the optimal physiological time point for inducing the male sterility. The varieties used did not show clear genotype-specific reactions to the treatments and a tenfold dose of CHA in a single application, i.e. a 10× concentration of CHA in potted plants with root application compared to field plants with spray application, proved to be very effective.

Example 2 shows that lower concentrations of CHA can be also effective, but they need to be applied over a longer time period.

Example 3 shows that CHA root treatment of wheat plants with adequate doses does not substantially harm the fertility of the female organs. After CHA-mediated emasculation and subsequent pollination with wheat pollen, the tested wheat varieties set morphologically normal seeds. Kernel numbers per spike were somewhat different between varieties, which confirms previous observations made with foliar CHA application.

1. A method of applying a CHA to a plant comprising the steps of:
(a) Providing a source comprising at least one CHA;
(b) Bringing said source in contact with at least one root of a plant; and
(c) Having the at least one root take up at least a part of said at least one CHA.

2. A method of inducing male sterility in a plant, comprising carrying out the steps according to claim 1, wherein said step (b) of bringing in contact is effected prior to flowering of said plant

3. A method of producing haploid embryos or plants comprising carrying out the steps according to claim 2, followed by:
(d) Fertilizing said plant with an appropriate pollen of a remote plant, thereby generating haploid embryos; and
(e) Optionally regenerating haploid plants from the haploid embryos generated in step (d).

4. Method A method of producing doubled-haploid plants comprising applying the steps according to the method of claim 3, followed by:
(f) Treatment of the plants with an agent causing doubling of the chromosome set, such as colchicine or nitrous oxide.

5. Method A method according to claim 4, which is for producing hybrid seed or a line variety.

6. A method of producing a plant, such as a parent plant for hybrid seed production comprising:
(g) Selecting from a pool of doubled-haploid plants produced by the method according to claim 4 a plant suitable as parent of a hybrid variety.

7. A doubled-haploid plant generated by the method according to claim 4.

8. A method of breeding comprising use of the plant obtained by the method of claim 3.

9. A method according to claim 1, wherein the plant is selected from cereals, fruits, vegetables, other crop plants and ornamentals.

10. A method according to claim 1, wherein said plant is a cereal plant.

11. A method according to claim 1, wherein said plant is selected from the group consisting of wheat spp. (Triticum spp.), rye, rice, barley, oats, millet and triticale.

12. A method according to claim 1, wherein said plant is wheat.

13. A method according to claim 1, wherein essentially no other plant part is in contact with said CHA.

14. Method A method according to claim 1, wherein the amount/concentration of CHA applied ranges between about 1.4 mg/plant and about 216 mg/plant.

15. A method according to claim 1, wherein said source is irrigation water.

16. A method according to claim 1, wherein said plant is cultivated in a substrate or as hydroponic culture.

17. A method according to claim 1, wherein said CHA is selected from clofencet, sintofen, azetidine-3-carboxylic acid, 2-chloroethylphosphonic acid (Ethrel), sodium 2,3-dichloroisobutyrate, triiodobenzoic acid, naphthalene acetic acid, maleic hydrazide, bromoxonil, glyphosate, giberrelic acid, iodosulfuron, flufenacet and nitroarylalkylsulfone derivatives and salts of any of the above.

18. A method according to claim 1, wherein CHA is selected from clofencet, sintofen, azetidine-3-carboxylic acid, sodium 2,3-dichloroisobutyrate, triiodobenzoic acid, naphthalene acetic acid, maleic hydrazide, bromoxonil, iodosulfuron, flufenacet and nitroarylalkylsulfone derivatives and salts of any of the above.

19. A method according to claim 1, wherein CHA is selected from clofencet, sintofen, azetidine-3-carboxylic acid, sodium 2,3-dichloroisobutyrate, triiodobenzoic acid, naphthalene acetic acid, maleic hydrazide, bromoxonil, iodosulfuron and nitroarylalkylsulfone derivatives and salts of any of the above.

20. A method according to claim 1, wherein CHA is selected from clofencet, sintofen or salts thereof.

21. A method according to claim 16, wherein for clofencet or azetidine-3-carboxylic acid when applied in cereals said contact with said CHA is between Zadoks stages 31 and 59.
